(19) United States
(12) Patent Application Publication
(10) Pub. No.: US 2004/0234483 A1
(43) Pub. Date: Nov. 25, 2004

(54) Clear personal care compositions containing a cationic conditioning polymer and an anionic surfactant system

(75) Inventors: Marjorie Mossman Peffly, Cincinnati, OH (US); Debora W. Chang, Mason, OH (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company

(21) Appl. No.: 10/851,356

(22) Filed: May 21, 2004

Related U.S. Application Data
(60) Provisional application No. 60/472,946, filed on May 23, 2003.

(51) Int. Cl. A61K 7/06; A61K 7/11

(52) U.S. Cl. 424/70.13

(57) ABSTRACT

The compositions of the present invention relate to personal cleansing compositions having from about 0.01 to about 5 wt. % of a cationic guar polymer; from about 5 to about 50 wt. % of an anionic surfactant system, having a specific ethoxylate level and sulfate level to optimize performance of the polymer, from about 0.01 to about 5 wt. % of a mono or divalent salt; from about 0.01% to about 10% of a microemulsified conditioning oil and at least about 20 wt. % of an aqueous carrier.

### CROSS REFERENCE TO RELATED APPLICATION

The application claims the benefit of U.S. Provisional application Ser. No. 60/472,946 (Case 9253P), filed on May 23, 2003.

### FIELD OF THE INVENTION

The present invention relates to a clear personal care composition containing a cationic conditioning polymer and a specific anionic surfactant system. More specifically, it relates to a personal care composition containing an anionic surfactant system with specific ratios of ethoxylate and sulfate such that when combined with the cationic conditioning polymer, specifically a guar, maximized levels of coacervate are achieved in use while maintaining excellent product clarity.

### BACKGROUND

Personal care compositions comprising various combinations of detersive surfactant and conditioning agents are known. These products typically comprise an anionic detersive surfactant in combination with a conditioning agent such as silicone, hydrocarbon oil, fatty esters, or combinations thereof. These products have become more popular among consumers as a means of conveniently obtaining hair and skin conditioning and cleansing performance all from a single personal care product.

However, many personal care compositions do not provide sufficient deposition of conditioning agents onto hair and skin during the cleansing process. Without such deposition, large proportions of conditioning agent are rinsed away during the cleansing process and therefore provide little or no conditioning benefit. Without sufficient deposition of the conditioning agent on the hair and skin, relatively high levels of conditioning agents may be needed in the personal cleansing composition to provide adequate conditioning performance. However, high levels of a conditioning agent can increase raw material costs, reduce lathering, and present product stability concerns as well as interfere with product clarity.

Obtaining good deposition of a conditioning agent is further complicated by the action of detersive surfactants in the personal care composition. Detersive surfactants are designed to carry away or remove oil, grease, dirt, and particulate matter from the hair and skin. In doing so, the detersive surfactants can also interfere with deposition of the conditioning agent, and both deposited and non deposited conditioning agent can be removed during rinsing. This further reduces deposition of the conditioning agent onto the hair and skin after rinsing, thus further reducing conditioning performance.

One known method for improving deposition of a conditioning agent involves the use of certain cationic deposition polymers. These polymers may be synthetic or natural cellulosic or guar polymers that have been modified with cationic substituents. The cationic charge density of such polymers, especially when used in a personal care composition, is minimized so as to avoid incompatibility with anionic materials in the product such as anionic surfactant. Thus, most personal care compositions that contain both an anionic detersive surfactant and a cationic deposition polymer have a relatively low level of coacervate conditioning. A need still exists for improved conditioning performance in personal care compositions.

### SUMMARY

The present invention meets the aforementioned need by providing a clear personal care composition comprising:

a. from about 0.01 to about 5 wt. % of a cationic guar polymer, wherein said cationic guar polymer has a molecular weight of at least 300,000 and a charge density of at least about 0.1 meq/g;

b. from about 5 to about 50 wt. % of an anionic surfactant system having an ethoxylate level and a sulfate level,

1. wherein said ethoxylate level is in the amount of about 1.97 multiplied by the molecular weight of said cationic guar polymer divided by 1,000,000 plus from about 0.15 to about 3.5, more preferably from about 0.4 to about 2.5,

2. wherein said sulfate level is in the amount of about 0.97 multiplied by the charge density of said cationic guar polymer plus from about 1 to about 4, more preferably from about 1.6 to about 3.5;

c. from about 0.01 to about 5 wt. % of a mono or divalent salt;

d. from about 0.01 to about 10 wt. % of a microemulsified conditioning oil having a droplet size of less than about 80 nm;

e. from about 0.05 to about 5% of a secondary conditioner; and

f. at least about 20 wt. % of an aqueous carrier;

wherein the composition, prior to the addition of any colorants and/or pigments, has a percent transmittance at 600 nm of at least about 65%.

The present invention is further directed to a method of using the personal care composition.

These and other features, aspects, and advantages of the present invention will become evident to those skilled in the art from a reading of the present disclosure.

### DETAILED DESCRIPTION

While the specification concludes with claims that particularly point out and distinctly claim the invention, it is believed the present invention will be better understood from the following description.

The personal care compositions of the present invention include a cationic guar polymer, an anionic surfactant system, a salt, a microemulsified conditioning oil and water. Each of these essential components, as well as preferred or optional components, are described in detail hereinafter.

All percentages, parts and ratios are based upon the total weight of the compositions of the present invention, unless otherwise specified. All such weights as they pertain to listed ingredients are based on the active level and, therefore, do not include solvents or by-products that may be included in commercially available materials, unless otherwise specified. The term “weight percent” may be denoted as “wt. %” herein.

All molecular weights as used herein are weight average molecular weights expressed as grams/mole, unless otherwise specified.

The term “charge density”, as used herein, refers to the ratio of the number of positive charges on a monomeric unit of which a polymer is comprised to the molecular weight of said monomeric unit. The charge density multiplied by the polymer molecular weight determines the number of positively charged sites on a given polymer chain.

Herein, “comprising” means that other steps and other ingredients which do not affect the end result can be added. This term encompasses the terms “consisting of” and “consisting essentially of”. The compositions and methods/processes of the present invention can comprise, consist of, and consist essentially of the essential elements and limitations of the invention described herein, as well as any of the additional or optional ingredients, components, steps, or limitations described herein.

The term “polymer” as used herein shall include materials whether made by polymerization of one type of monomer or made by two (i.e., copolymers) or more types of monomers.

The term “solid particle” as used herein means a particle that is not a liquid or a gas.

The term “water soluble” as used herein, means that the polymer is soluble in water in the present composition. In general, the polymer should be soluble at 25° C. at a concentration of 0.1% by weight of the water solvent, preferably at 1%, more preferably at 5%, most preferably at 15%.

The term “water insoluble” as used herein, means that the polymer is not soluble in water in the present composition. Thus, the polymer is not miscible with water.

The term “clear” as used herein, means that the finished product excluding any colorants that may be used has a % transmittance at 600 nm of at least about 65%.

The % transmittance of the composition is measured using Ultra-Violet/Visible (UV/VIS) Spectrophotometry, which determines the absorption or transmission of UV/VIS light by a sample. A light wavelength of 600 nm has been shown to be adequate for characterizing the degree of clarity of cosmetic compositions. Typically, it is best to follow the specific instructions for the specific spectrophotometer being used. In general, the procedure for measuring percent transmittance starts by setting the spectrophotometer to the 600 nm. Then a calibration “blank” is run to calibrate the readout to 100 percent transmittance. The test sample is then placed in a cuvette designed to fit the specific spectrophotomer and the percent transmittance is measured by the spectrophotomer at 600 nm.

All cited references are incorporated herein by reference in their entireties. Citation of any reference is not an admission regarding any determination as to its availability as prior art to the claimed invention.

One embodiment of the present invention concerns the surprising discovery that compositions combining certain specific levels and ratios of anionic surfactant as described by the sulfate and ethoxylation values (described herein) maximize the conditioning benefit via maximization of coacervate formation and maximize product clarity. Moreover, while the optimum surfactant blend is different for each polymer, the authors have discovered that the optimum surfactant composition can be described by two parameters. These parameters include sulfate and ethoxylation values that when expressed as a function of the polymer's charge density and molecular weight maximize the formation of coacervate and product clarity.

Coacervates, without being limited to a particular theory, provide improved hair and skin conditioning without any additional conditioning actives. Further, when dispersed conditioning agent droplets are added to the matrix, the coacervate provides an improved mechanism for conditioning agent deposition, yielding conditioning agent deposition that results in even more of a conditioning benefit.

A. Cationic Conditioning Polymer

The composition of the invention comprises a cationic conditioning polymer, which is a cationic derivative of a guar gum.

Suitable cationic guar gum derivatives are those given the CTFA designation guar hydroxypropyl trimonium chloride, available commercially for example as N-Hance 3205, which has a low degree of substitution of the cationic groups and a high viscosity. The low degree of cationic substitution leads to a cationic charge density of about 0.8 meq/g. Other suitable materials available from Aqualon include, but are not limited to, N-Hance 3198, which has a low degree of substitution and a high viscosity, N-Hance 3215, which has a moderate degree of substitution and a high viscosity, and N-Hance 3269 which has a low degree of substitution and a moderate viscosity. Also suitable are guar polymers available from Rhodia which include, but are not limited to, Jaguar C14S, having a low degree of substitution and a high viscosity, Jaguar CHT, having a high transparency, a moderate degree of substitution and a high viscosity, Jaguar C17 (low degree of substitution, high viscosity) and Jaguar C162 which is a hydroxypropylated cationic guar derivative containing a low level of substituent groups as well as cationic quaternary ammonium groups. Jaguar C162 has a cationic charge density of 0.8 meq/g.

The compositions of the invention contain from about 0.01% to about 5% by weight of cationic conditioning polymer, preferably from about 0.1% to about 2% by weight.

B. Anionic Surfactant System—Ethoxylate and Sulfate

The personal care composition of the present invention includes an anionic surfactant. The surfactant component is included to provide cleaning performance to the composition. The surfactant component in turn comprises an ethoxylated surfactant and a sulfate, and optionally a zwitterionic or amphoteric surfactant, an additional surfactant, or a combination thereof. Such surfactants should be physically and chemically compatible with the essential components described herein, or should not otherwise unduly impair product stability, aesthetics or performance.

Suitable anionic surfactant components for use in the personal care composition herein include those that are known for use in hair care or other personal care compositions. The concentration of the anionic surfactant component in the personal care composition should be sufficient to provide the desired cleaning and lather performance, and generally range from about 5% to about 50%, preferably from about 8% to about 30%, more preferably from about 10% to about 25%, by weight of the composition.

In considering the performance characteristics of product clarity, coacervate generation, wet conditioning performance, dry conditioning performance, and conditioning ingredient deposition on hair, the present inventors discovered that in order to maximize the performance potential of polymer systems, it is necessary to optimize the levels and types of surfactant. The present inventors discovered that in order to maximize the performance potential of polymer systems, it is necessary to optimize the levels of ethoxylate and sulfate. In order to do this, the aforementioned performance characteristics were plotted versus ethoxylate levels and versus sulfate levels. The “peak” or optimal level of sulfate and ethoxylate was then plotted versus a variety of chemical descriptors for cationic polymeric structures including molecular weight, and charge density.

The present inventors discovered that clear correlations exist when the optimal level of ethoxylate providing peak performance was plotted as the “y” axis versus polymer molecular weight divided by 1,000,000 as the “x” axis. The equation of that line showed that the slope of the line was equal to approximately 1.97 and the intercept fell between from about 0.15 to about 3.5. Preferably the intercept is greater than 0.15, more preferably greater than 0.4. Preferably the intercept is less than 3.5, more preferably less than 2.5. Thus, for any given molecular weight cationic guar polymer, an optimum ethoxylate level could be calculated. Additionally, a second correlation was developed where the optimal level of sulfate was plotted as the “y” axis and charge density was plotted as the “x” axis. Again, a relatively straight line was realized where the slope of the line was equal to approximately 0.97 and the intercept fell between from about 1 to about 4. Preferably, the intercept is greater than 1, more preferably greater than 1.6. Preferably, the intercept is less than 4, more preferably less than 3.5.

Thus, a percent ethoxylate can be calculated based on the stochiometry of the surfactant structure, based on a particular molecular weight of the surfactant where the number of moles of ethoxylation is known. Likewise, given a specific molecular weight of a surfactant and a sulfation reaction completion measurement, the percent sulfate can be calculated. Analytical techniques have been developed to measure percent ethoxylation or percent sulfates within surfactant systems. The level of ethoxylate and the level of sulfate representative of a particular surfactant system is calculated from the percent ethoxylation and percent sulfates of individual surfactants in the following manner:

Level of Ethoxylate in a composition=percent ethoxylation multiplied by percent active ethoxylated surfactant.

Level of Sulfate in a composition=percent Sulfate in ethoxylated surfactant multiplied by percent active ethoxylated surfactant plus percent sulfate in non-ethoxylated surfactant multiplied by percent active non-ethoxylated surfactant.

Sample Calculation:

Example 1 shows an ethoxylated surfactant that contains 0.294321% ethoxylate and a 0.188307% sulfate level and a non-ethoxylated surfactant with a percent sulfate of 0.266845. Both surfactants are 29% active.

Level of Ethoxylate in Example 1=0.294321 multiplied by 8.5 (% active ethoxylated surfactant). Thus the level of ethoxylate in Example 1's composition is approximately 2.5.

Level of Sulfate in Example 1=0.18830 multiplied by 8.5 (% active ethoxylated surfactant) plus 0.266845 multiplied by 5.5 (% active non-ethoxylated surfactant). Thus the level of sulfate in Example 1's composition is approximately 3.07.

Preferred anionic surfactants suitable for use in the personal care compositions are the alkyl sulfates and alkyl ether sulfates. These materials have the respective formulae ROSO₃M and RO(C₂H₄O)ₓSO₃M, wherein R is alkyl or alkenyl of from about 8 to about 18 carbon atoms, x is an integer having a value of from about 1 to about 10, and M is a cation such as ammonium, alkanolamines, such as triethanolamine, monovalent metals, such as sodium and potassium, and polyvalent metal cations, such as magnesium, and calcium. Solubility of the surfactant will depend upon the particular anionic surfactants and cations chosen.

Preferably, R has from about 8 to about 18 carbon atoms, more preferably from about 10 to about 16 carbon atoms, even more preferably from about 12 to about 14 carbon atoms, in both the alkyl sulfates and alkyl ether sulfates. The alkyl ether sulfates are typically made as condensation products of ethylene oxide and monohydric alcohols having from about 8 to about 24 carbon atoms. The alcohols can be synthetic or they can be derived from fats, e.g., coconut oil, palm kernel oil, tallow. Lauryl alcohol and straight chain alcohols derived from coconut oil or palm kernel oil are preferred. Such alcohols are reacted with between from about 0 and about 10, preferably from about 2 to about 5, more preferably from about 3, molar proportions of ethylene oxide, and the resulting mixture of molecular species having, for example, an average of about 3 moles of ethylene oxide per mole of alcohol, is sulfated and neutralized.

Specific non-limiting examples of alkyl ether sulfates which may be used in the personal care compositions of the present invention include sodium and ammonium salts of coconut alkyl triethylene glycol ether sulfate, tallow alkyl triethylene glycol ether sulfate, and tallow alkyl hexa-oxyethylene sulfate. Highly preferred alkyl ether sulfates are those comprising a mixture of individual compounds, wherein the compounds in the mixture have an average alkyl chain length of from about 10 to about 16 carbon atoms and an average degree of ethoxylation of from about 1 to about 4 moles of ethylene oxide. Such a mixture also comprises from about 0 to about 20% by weight C₁₂₋₁₃ compounds; from about 60 to about 100% by weight of C₁₄₋₁₅₋₁₆ compounds; from about 0 to about 20% by weight of C₁₇₋₁₈₋₁₉ compounds; from about 3 to about 30% by weight of compounds having a degree of ethoxylation of 0; from about 45 to about 90% by weight of compounds having a degree of ethoxylation of from about 1 to about 4; from about 10 to about 25% by weight of compounds having a degree of ethoxylation of from about 4 to about 8; and from about 0.1 to about 15% by weight of compounds having a degree of ethoxylation greater than about 8.

Another suitable class of anionic surfactants are the water-soluble salts of the organic, sulfuric acid reaction products of the general formula R₁—SO₃—M wherein R₁ is chosen from the group consisting of a straight or branched chain, saturated aliphatic hydrocarbon radical having from about 8 to about 24, preferably about 12 to about 18, carbon atoms; and M is a cation. Important examples are the salts of an organic sulfuric acid reaction product of a hydrocarbon of the methane series, including iso-, neo-, ineso-, and n-paraffins, having about 8 to about 24 carbon atoms, preferably about 12 to about 18 carbon atoms, and a sulfonating agent e.g., SO₃, H₂SO₄, oleum, obtained according to known sulfonation methods, including bleaching and hydrolysis. Preferred are alkali metal and ammonium sulfonated C₁₂₋₁₈-n-paraffins.

Preferred anionic surfactants for use in the personal care compositions include ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium lauryl sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, and combinations thereof.

C. Mono or Divalent Salt

The personal care compositions of the present invention further comprise a mono or divalent salt, which acts as a source of entropy in order to act as a coacervate initiator. Salt allows more contacts to be made between the polymer and surfactant, which increases the formation of coacervates. The term “coacervate initiator”, as used herein, means electrolytes capable of inducing the formation of coacervates when combined with compositions comprising an anionic detersive surfactant component surfactant system and the synthetic cationic polymer.

Surfactant salts themselves are not included in the present electrolyte definition but other salts are. Suitable salts include, but are not limited to chlorides, phosphates, sulfates, nitrates, citrates and halides. The counter ions of such salts can be, but are not limited to, sodium, potassium, ammonium, magnesium, zinc or other mono and divalent cation. Electrolytes most preferred for use in the compositions of the present invention include sodium chloride, ammonium chloride, sodium citrate, magnesium chloride, and magnesium sulfate. It is recognized that these salts may serve as thickening aids or buffering aids in addition to their role as a coacervate initiator. The amount of coacervate initiator comprising the electrolyte and/or the optional surfactant will vary with the type of surfactant and polymer, but is preferably present at a level of from about 0.01% to about 5%, more preferably from about 0.05% to about 3.5%, and still more preferably from about 0.1% to about 2%.

D. Microemulsified Conditioning Oil

The personal care compositions of the present invention further comprise a microemulsified conditioning oil. These include materials used to give a particular conditioning benefit to hair and/or skin. The conditioning agents useful in the personal care compositions of the present invention typically comprise a water insoluble, water dispersible, non-volatile, liquid that forms emulsified, liquid droplets in a surfactant component. Suitable conditioning agents for use in the personal care composition are those conditioning agents characterized generally as silicone oils, organic conditioning oils (e.g. hydrocarbon oils, polyolefins, fatty esters, and fatty alcohols), fluorinated compounds, or combinations thereof, or those conditioning agents which otherwise form liquid, dispersed, droplets in the aqueous surfactant matrix herein. Such conditioning agents should be physically and chemically compatible with the essential components of the composition, and should not otherwise unduly impair product stability, aesthetics or performance.

The concentration of the conditioning agent in the personal care composition should be sufficient to provide the desired conditioning benefits, as will be apparent to one of ordinary skill in the art. Such concentration can vary with the conditioning agent, the conditioning performance desired, the average size of the conditioning agent droplets, the type and concentration of other components, and other like factors. The concentration of the conditioning agent may range from about 0.01 to about 10 weight percent and have a droplet size of less than about 80 nm, preferably less than about 50 nm.

a. Silicone Oils

Silicone oils are typically flowable silicone materials having a viscosity, as measured at 25° C., less than 1,000,000 centistokes (cst), preferably from about 5 cst to about 1,000,000 cst, more preferably from about 10 cst to about 200,000 cst. Suitable silicone oils for use in the personal care compositions of the present invention include polyalkyl siloxanes, polyaryl siloxanes, polyalkylaryl siloxanes, polyether siloxane copolymers, and mixtures thereof. Other insoluble, non-volatile silicone fluids having hair conditioning properties may also be used.

Silicone oils include polyalkyl or polyaryl siloxanes which conform to the following Formula I:

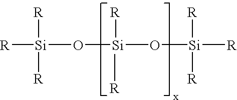

wherein R is aliphatic, preferably alkyl or alkenyl, or aryl, R can be substituted or unsubstituted, and x is an integer from 1 to about 8,000. Suitable unsubstituted R groups for use in the personal care compositions of the present invention include, but are not limited to: alkoxy, aryloxy, alkaryl, arylalkyl, arylalkenyl, alkamino, and ether-substituted, hydroxyl-substituted, and halogen-substituted aliphatic and aryl groups. Suitable R groups also include cationic amines and quaternary ammonium groups.

Preferred alkyl and alkenyl substituents are C₁ to C₅ alkyls and alkenyls, more preferably from C₁ to C₄, more preferably from C₁ to C₂. The aliphatic portions of other alkyl-, alkenyl-, or alkynyl-containing groups (such as alkoxy, alkaryl, and alkamino) can be straight or branched chains, and are preferably from C₁ to C₅, more preferably from C₁ to C₄, even more preferably from C₁ to C₃, more preferably from C₁ to C₂. As discussed above, the R substituents can also contain amino functionalities (e.g. alkamino groups), which can be primary, secondary or tertiary amines or quaternary ammonium. These include mono-, di- and tri-alkylamino and alkoxyamino groups, wherein the aliphatic portion chain length is described above. Examples of silicone microemulsions consistent with the present invention include DC2-5791, DC2-5791-sp, DC2-1470, DC2-1870, DC2-1845, DC2-1845HV, and DC2-1550 from Dow Corning. In general, the surfactant system used in the microemulsion must be compatible with the guar polymer and other surfactants present in the composition of the present invention.

b. Organic Conditioning Oils

The microemulsified conditioning oil of the personal care compositions of the present invention may also comprise from about 0.05% to about 3%, by weight of the composition, preferably from about 0.08% to about 1.5%, more preferably from about 0.1% to about 1%, of at least one organic conditioning oil, either alone or in combination with other conditioning agents, such as the silicone oils (described above).

i. Hydrocarbon Oils

Suitable organic conditioning oils for use as conditioning agents in the personal care compositions of the present invention include, but are not limited to, hydrocarbon oils having at least about 10 carbon atoms, such as cyclic hydrocarbons, straight chain aliphatic hydrocarbons (saturated or unsaturated), and branched chain aliphatic hydrocarbons (saturated or unsaturated), including polymers and mixtures thereof. Straight chain hydrocarbon oils preferably are from about C₁₂ to about C₁₉. Branched chain hydrocarbon oils, including hydrocarbon polymers, typically will contain more than 19 carbon atoms.

Specific non-limiting examples of these hydrocarbon oils include paraffin oil, mineral oil, saturated and unsaturated dodecane, saturated and unsaturated tridecane, saturated and unsaturated tetradecane, saturated and unsaturated pentadecane, saturated and unsaturated hexadecane, polybutene, polydecene, and mixtures thereof. Branched-chain isomers of these compounds, as well as of higher chain length hydrocarbons, can also be used, examples of which include highly branched, saturated or unsaturated, alkanes such as the permethyl-substituted isomers, e.g., the permethyl-substituted isomers of hexadecane and eicosane, such as 2,2,4,4,6,6,8,8-dimethyl-10-methylundecane and 2,2,4,4,6,6-dimethyl-8-methylnonane, available from Permethyl Corporation. Hydrocarbon polymers such as polybutene and polydecene. A preferred hydrocarbon polymer is polybutene, such as the copolymer of isobutylene and butene. A commercially available material of this type is L-14 polybutene from Amoco Chemical Corporation.

ii. Polyolefins

Organic conditioning oils for use in the personal care compositions of the present invention can also include liquid polyolefins, more preferably liquid poly-α-olefins, more preferably hydrogenated liquid poly-α-olefins. Polyolefins for use herein are prepared by polymerization of C₄ to about C₁₄ olefenic monomers, preferably from about C₆ to about C₁₂.

Non-limiting examples of olefenic monomers for use in preparing the polyolefin liquids herein include ethylene, propylene, 1-butene, 1-pentene, 1-hexene, 1-octene, 1-decene, 1-dodecene, 1-tetradecene, branched chain isomers such as 4-methyl-1-pentene, and mixtures thereof. Also suitable for preparing the polyolefin liquids are olefin-containing refinery feedstocks or effluents. Preferred hydrogenated α-olefin monomers include, but are not limited to: 1-hexene to 1-hexadecenes, 1-octene to 1-tetradecene, and mixtures thereof.

iii. Fatty Esters

Other suitable organic conditioning oils for use as the conditioning agent in the personal care compositions of the present invention include, but are not limited to, fatty esters having at least 10 carbon atoms. These fatty esters include esters with hydrocarbyl chains derived from fatty acids or alcohols (e.g. mono-esters, polyhydric alcohol esters, and di- and tri-carboxylic acid esters). The hydrocarbyl radicals of the fatty esters hereof may include or have covalently bonded thereto other compatible functionalities, such as amides and alkoxy moieties (e.g., ethoxy or ether linkages, etc.).

Specific examples of preferred fatty esters include, but are not limited to: isopropyl isostearate, hexyl laurate, isohexyl laurate, isohexyl palmitate, isopropyl palmitate, decyl oleate, isodecyl oleate, hexadecyl stearate, decyl stearate, isopropyl isostearate, dihexyldecyl adipate, lauryl lactate, myristyl lactate, cetyl lactate, oleyl stearate, oleyl oleate, oleyl myristate, lauryl acetate, cetyl propionate, and oleyl adipate.

Other fatty esters suitable for use in the personal care compositions of the present invention are mono-carboxylic acid esters of the general formula R′COOR, wherein R′ and R are alkyl or alkenyl radicals, and the sum of carbon atoms in R′ and R is at least 10, preferably at least 22.

Still other fatty esters suitable for use in the personal care compositions of the present invention are di- and tri-alkyl and alkenyl esters of carboxylic acids, such as esters of C₄ to C₈ dicarboxylic acids (e.g. C₁ to C₂₂ esters, preferably C₁ to C₆, of succinic acid, glutaric acid, adipic acid,). Specific non-limiting examples of di- and tri-alkyl and alkenyl esters of carboxylic acids include isocetyl stearyol stearate, diisopropyl adipate, and tristearyl citrate.

Other fatty esters suitable for use in the personal care compositions of the present invention are those known as polyhydric alcohol esters. Such polyhydric alcohol esters include alkylene glycol esters, such as ethylene glycol mono and di-fatty acid esters, diethylene glycol mono- and di-fatty acid esters, polyethylene glycol mono- and di-fatty acid esters, propylene glycol mono- and di-fatty acid esters, polypropylene glycol monooleate, polypropylene glycol 2000 monostearate, ethoxylated propylene glycol monostearate, glyceryl mono- and di-fatty acid esters, polyglycerol poly-fatty acid esters, ethoxylated glyceryl monostearate, 1,3-butylene glycol monostearate, 1,3-butylene glycol distearate, polyoxyethylene polyol fatty acid ester, sorbitan fatty acid esters, and polyoxyethylene sorbitan fatty acid esters.

Still other fatty esters suitable for use in the personal care compositions of the present invention are glycerides, including, but not limited to, mono-, di-, and tri-glycerides, preferably di- and tri-glycerides, more preferably triglycerides. For use in the personal care compositions described herein, the glycerides are preferably the mono-, di-, and tri-esters of glycerol and long chain carboxylic acids, such as C₁₀ to C₂₂ carboxylic acids. A variety of these types of materials can be obtained from vegetable and animal fats and oils, such as castor oil, safflower oil, cottonseed oil, corn oil, olive oil, cod liver oil, almond oil, avocado oil, palm oil, sesame oil, lanolin and soybean oil. Synthetic oils include, but are not limited to, triolein and tristearin glyceryl dilaurate.

Other fatty esters suitable for use in the personal care compositions of the present invention are water insoluble synthetic fatty esters. Some preferred synthetic esters conform to the general Formula II:

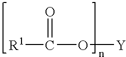

wherein R¹ is a C₇ to C₉ alkyl, alkenyl, hydroxyalkyl or hydroxyalkenyl group, preferably a saturated alkyl group, more preferably a saturated, linear, alkyl group; n is a positive integer having a value from 2 to 4, preferably 3; and Y is an alkyl, alkenyl, hydroxy or carboxy substituted alkyl or alkenyl, having from about 2 to about 20 carbon atoms, preferably from about 3 to about 14 carbon atoms. Other preferred synthetic esters conform to the general Formula III:

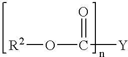

wherein R² is a C₈ to C₁₀ alkyl, alkenyl, hydroxyalkyl or hydroxyalkenyl group; preferably a saturated alkyl group, more preferably a saturated, linear, alkyl group; n and Y are as defined above in Formula III.

Specific non-limiting examples of suitable synthetic fatty esters for use in the personal care compositions of the present invention include: P-43 (C₈-C₁₀ triester of trimethylolpropane), MCP-684 (tetraester of 3,3 diethanol-1,5 pentadiol), MCP 121 (C₈-C₁₀ diester of adipic acid), all of which are available from Mobil Chemical Company.

c. Fluorinated Compounds

Fluorinated compounds suitable for delivering conditioning to hair or skin consistent with the present invention include perfluoropolyethers, perfluorinated olefins, fluorine based specialty polymers that may be in a fluid or elastomer form similar to the silicone fluids previously described, and perfluorinated dimethicones Specific non-limiting examples of suitable fluorinated compounds include the Fomblin product line from Ausimont which include HC/04, HC/25, HC01, HC/02, HC/03; Dioctyldodecyl Fluoroeptyl Citrate, commonly called Biosil Basics Fluoro Gerbet 3.5 supplied by Biosil Technologies; and Biosil Basics Fluorosil LF also supplied by Biosil Technologies.

E. Secondary Conditioners

The personal care compositions of the present invention further contain secondary conditioning ingredients. These include materials used to provide additional conditioning benefits to hair and/or skin beyond that provided by the microemulsified conditioning oil. The secondary conditioning ingredients used in the compositions of the present invention typically comprise both water soluble materials and water insoluble, water dispersible, non-volatile materials that can be solubilized by the surfactant micelles of the anionic surfactant component. Suitable secondary conditioning ingredients for use in the personal care composition are those materials generally characterized as quaternary ammonium compounds, organic conditioning compounds (e.g. fatty esters, fatty alcohols, polyethylene glycols, alkyl glucosides and alkyl glucoside derivatives), or combinations thereof. Such secondary conditioning ingredients should be physically and chemically compatible with the essential components of the composition, and should not otherwise unduly impair product stability, aesthetics or performance. The secondary conditioning component of the personal care compositions of the present invention comprises from about 0.05% to about 5%, by weight of the composition, preferably from about 0.1% to about 3%, more preferably from about 0.5% to about 2%.

a. Quaternary Ammonium Compounds

Suitable quaternary ammonium compounds for use as secondary conditioning ingredients in the personal care compositions of the present invention include, but are not limited to, hydrophilic quaternary ammonium compounds with a long chain substituent having a carbonyl moiety, like an amide moiety, or a phosphate ester moiety or a similar hydrophilic moiety.

In particular, one class of hydrophilic quaternary ammonium compounds found useful in the composition of the present invention is depicted by general structural formula (IV):

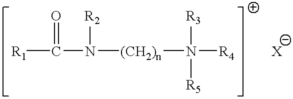

wherein R₁ is a substituted or unsubstituted, saturated or unsaturated, alkyl group including about 5 to about 21 carbon atoms; R₂ is hydrogen or methyl; R₃ and R₄, independently, are methyl, ethyl, hydroxyethyl or benzyl; R₅ is methyl, ethyl, hydroxyethyl, benzyl, acetamido or 2-pyrrolidonyl; n is a numeral from one to about 10; and X is an anion selected from the group consisting of chloride, bromide, ethosulfate, methosulfate, acetate, nitrate, tosylate, phosphate, and combinations thereof.

Examples of useful hydrophilic quaternary ammonium compounds having the general structural formula (IV) include, but are not limited to, compounds designated in the CTFA Cosmetic Dictionary, as ricinoleamidopropyl trimonium chloride, ricinoleamido trimonium ethylsulfate, hydroxy stearamidopropyl trimoniummethylsulfate and hydroxy stearamidopropyl trimonium chloride, or combinations thereof.

Examples of other useful quaternary ammonium surfactants include, but are not limited to, Quaternium-33, Quaternium-43, isostearamidopropyl ethyldimonium ethosulfate, Quaternium-22 and Quaternium-26, or combinations thereof, as designated in the CTFA Dictionary.

Another particularly useful class of quaternary ammonium compounds that can be included in the composition of the present invention are the quaternized phosphate esters, as depicted in general structural formula (V):

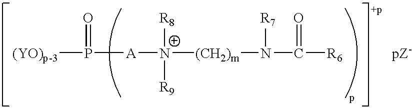

wherein R₆ is an aryl, an alkaryl, a saturated or unsaturated alkyl group, or a saturated or unsaturated hydroxyalkyl group, wherein the alkyl or hydroxyalkyl group includes about seven to about 21 carbon atoms; R₇ is hydrogen, or an alkyl or a hydroxyalkyl group including one to about six carbon atoms; R₈ and R₉, independently, are an alkyl or a hydroxyalkyl group including one to about six carbon atoms; A is a residue of a glycol or a triol having two to about four carbon atoms, such as the residue of propylene glycol (—OCH₂ CH(OH)CH₂—); Z is an anion selected from the group consisting of chloride, bromide, methosulfate, ethosulfate, and combinations thereof; m is a numeral from one to about 10; Y is selected from the group consisting of hydrogen, an alkyl group, a hydroxyalkyl group, and an aryl group, either substituted or unsubstituted, and wherein the alkyl or the hydroxyalkyl group includes one to about 22 carbon atoms; and p is a number from 1 to 3. To achieve the full advantage of the present invention, the quaternized phosphate ester is a quaternized phosphate diester that includes the alkyl moiety of an essential fatty acid, like linoleic acid, arachidonic acid or ricinoleic acid, as the R₆ substituent of the compound. For example, the quaternized phosphate ester of general structural formula (IV) that includes the alkyl moiety of an essential fatty acid as the R₆ substituent and wherein the number p is 2.

The essential fatty acid substituent helps the compound impart conditioning properties to the hair and skin. An example of an especially useful quaternized phosphate diester is depicted in structural formula (VI),

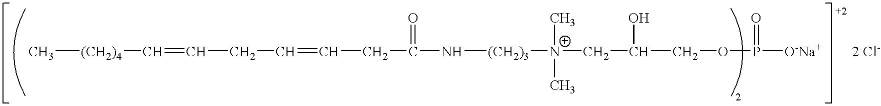

available commercially under the brandname PHOSPHOLIPID EFA, from Uniqema, and having the proposed CTFA Dictionary designation linoleamidopropyl PG-dimonium chloride phosphate. This particular compound has p equal to 2 and includes the alkyl moiety of linoleic acid as the substituent R₆.

It should be understood that the monophosphate ester (i.e., p=1) and triphosphate ester (i.e., p=3) of the quaternized phosphate ester illustrated in general structural formula (VI) also can be used in the composition of the present invention as long as the basic properties of the conditioning shampoo are not adversely affected.

Other hydrophilic quaternary ammonium compounds useful in a composition of the present invention, and that do not belong to the two classes of hydrophilic quaternary ammonium compounds described above include, but are not limited to, Quaternium-16, Quaternium-27, Quaternium-30, Quaternium-52, Quaternium-53, Quaternium-56, Quaternium-60, Quaternium-61, Quaternium-62, Quaternium-63, Quaternium-71, and combinations thereof.

b. Organic Conditioning Compounds

i. Fatty Esters

Suitable organic compounds for use as secondary conditioning ingredients in the compositions of the present invention include, but are not limited to, fatty esters such as those described under the Microemulsified conditioning oils section above. In particular, one class of fatty esters found extremely useful in the composition of the present invention when used as a secondary conditioner, includes fatty alkoxylate esters of aliphatic or aromatic, dicarboxylic or tricarboxylic acids. Examples of suitable aliphatic dicarboxylic acids include, but are not limited to, malonic acid, succinic acid, maleic acid, and adipic acid. The fatty alkoxylate esters are formed by reacting dicarboxylic and tricarboxylic acids with polyalkoxylated fatty alcohols. Saturated, unsubstituted aliphatic moieties are preferred for the diesters of dicarboxylic acids, and such fatty moieties containing from 14 to 18 carbon atoms are more preferred. An example of an especially useful fatty alkoxylate ester is available commercially under the brandname Cromollient SCE, from Croda, Inc. Other suitable fatty alkoxylate esters are disclosed in U.S. Pat. No. 5,455,025 to Croda.

ii. Fatty Alcohols

Other suitable organic compounds for use in the personal care compositions of the present invention include, but are not limited to, fatty alcohols having at least 10 carbon atoms, more preferably 10 to 22 carbon atoms, most preferably 12 to 16 carbon atoms. Another class of fatty alcohols suitable for use in the personal care compositions of the present inventions conforms to the general Formula VII:

CH₃(CH₂)ₙCH₂(OCH₂CH₂)ₚOH

wherein n is a positive integer having a value from 8 to 20, preferably 10 to 14, and p is a positive integer having a value from 1 to 30, preferably from 2 to 23.

iii. Polyethylene Glycols

Additional organic compounds useful herein include polyethylene glycols and polypropylene glycols having a molecular weight of up to about 2,000,000 such as those with CTFA names PEG-200, PEG-400, PEG-600, PEG-1000, PEG-2M, PEG-7M, PEG-14M, PEG-45M and mixtures thereof.

iv. Alkyl Glucosides and Alkyl Glucoside Derivatives

Suitable organic compounds for use in the personal care compositions of the present invention include, but are not limited to, alkyl glucosides and alkyl glucoside derivatives. Specific non-limiting examples of suitable alkyl glucosides and alkyl glucoside derivatives include Glucam E-10, Glucam E-20, Glucam P-10, and Glucquat 125 commercially available from Amerchol.

F. Aqueous Carrier

The compositions of the present invention include an aqueous carrier. The level and species of the carrier are selected according to the compatibility with other components and other desired characteristic of the product.

Carriers useful in the present invention include water and water solutions of lower alkyl alcohols. Lower alkyl alcohols useful herein are monohydric alcohols having 1 to 6 carbons, more preferably ethanol and isopropanol.

Preferably, the aqueous carrier is substantially water. Deionized water is preferably used. Water from natural sources containing mineral cations can also be used, depending on the desired characteristic of the product. Generally, the compositions of the present invention comprise from at least about 20% to about 99%, preferably from about 40% to about 98%, and more preferably from about 60% to about 98% aqueous carrier.

The pH of the present composition is preferably from about 4 to about 9, more preferably from about 4.5 to about 7.5. Buffers and other pH adjusting agents can be included to achieve the desirable pH.

G. Surfactant Coacervate Enhancers

1. Zwitterionic or Amphoteric Surfactant

Suitable amphoteric or zwitterionic surfactants for use in the personal care composition herein include those which are known for use in hair care or other personal care compositions. Concentration of such amphoteric surfactants preferably ranges from about 0.5% to about 20%, preferably from about 1% to about 10%, by weight of the composition. Non limiting examples of suitable zwitterionic or amphoteric surfactants are described in U.S. Pat. No. 5,104,646 (Bolich Jr. et al.), U.S. Pat. No. 5,106,609 (Bolich Jr. et al.).

Amphoteric surfactants suitable for use in the personal care composition are well known in the art, and include those surfactants broadly described as derivatives of aliphatic secondary and tertiary amines in which the aliphatic radical can be straight or branched chain and wherein one of the aliphatic substituents contains from about 8 to about 18 carbon atoms and one contains an anionic water solubilizing group such as carboxy, sulfonate, sulfate, phosphate, or phosphonate. Preferred amphoteric surfactants for use in the present invention include cocoamphoacetate, cocoamphodiacetate, lauroamphoacetate, lauroamphodiacetate, and mixtures thereof.

Zwitterionic surfactants suitable for use in the personal care composition are well known in the art, and include those surfactants broadly described as derivatives of aliphatic quaternary ammonium, phosphonium, and sulfonium compounds, in which the aliphatic radicals can be straight or branched chain, and wherein one of the aliphatic substituents contains from about 8 to about 18 carbon atoms and one contains an anionic group such as carboxy, sulfonate, sulfate, phosphate or phosphonate. Zwitterionics such as imadazolines are preferred.

2. Optional Surfactants

The personal care compositions of the present invention may further comprise additional surfactants for use in combination with the surfactant component described hereinbefore. Other suitable anionic surfactants are the water-soluble salts of organic, sulfuric acid reaction products conforming to the formula [R¹—SO₃—M] where R¹ is a straight or branched chain, saturated, aliphatic hydrocarbon radical having from about 8 to about 24, preferably from about 10 to about 18, carbon atoms; and M is a cation described hereinbefore. Non limiting examples of such surfactants are the salts of an organic sulfuric acid reaction product of a hydrocarbon of the methane series, including iso-, neo-, and n-paraffins, having from about 8 to about 24 carbon atoms, preferably about 12 to about 18 carbon atoms and a sulfonating agent, e.g., SO₃, H₂SO₄, obtained according to known sulfonation methods, including bleaching and hydrolysis. Preferred are alkali metal and ammonium sulfonated C₁₀ to C₁₈ n-paraffins.

Still other suitable anionic surfactants are the reaction products of fatty acids esterified with isethionic acid and neutralized with sodium hydroxide where, for example, the fatty acids are derived from coconut oil or palm kernel oil; sodium or potassium salts of fatty acid amides of methyl tauride in which the fatty acids, for example, are derived from coconut oil or palm kernel oil. Other similar anionic surfactants are described in U.S. Pat. Nos. 2,486,921; 2,486,922; and 2,396,278.

Other anionic surfactants suitable for use in the personal care compositions are the succinnates, examples of which include disodium N-octadecylsulfosuccinnate; disodium lauryl sulfosuccinate; diammonium lauryl sulfosuccinate; tetrasodium N-(1,2-dicarboxyethyl)-N-octadecylsulfosuccinnate; diamyl ester of sodium sulfosuccinic acid; dihexyl ester of sodium sulfosuccinic acid; and dioctyl esters of sodium sulfosuccinic acid.

Other suitable anionic surfactants include olefin sulfonates having about 10 to about 24 carbon atoms. In this context, the term “olefin sulfonates” refers to compounds which can be produced by the sulfonation of alpha-olefins by means of uncomplexed sulfur trioxide, followed by neutralization of the acid reaction mixture in conditions such that any sulfonates which have been formed in the reaction are hydrolyzed to give the corresponding hydroxy-alkanesulfonates. The sulfur trioxide can be liquid or gaseous, and is usually, but not necessarily, diluted by inert diluents, for example by liquid SO₂, chlorinated hydrocarbons, etc., when used in the liquid form, or by air, nitrogen, gaseous SO₂, etc., when used in the gaseous form. The alpha-olefins from which the olefin sulfonates are derived are mono-olefins having from about 10 to about 24 carbon atoms, preferably from about 12 to about 16 carbon atoms. Preferably, they are straight chain olefins. In addition to the true alkene sulfonates and a proportion of hydroxy-alkanesulfonates, the olefin sulfonates can contain minor amounts of other materials, such as alkene disulfonates depending upon the reaction conditions, proportion of reactants, the nature of the starting olefins and impurities in the olefin stock and side reactions during the sulfonation process. A non limiting example of such an alpha-olefin sulfonate mixture is described in U.S. Pat. No. 3,332,880.

Another class of anionic surfactants suitable for use in the personal care compositions are the beta-alkyloxy alkane sulfonates. These surfactants conform to the Formula VIII:

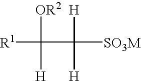

where R¹ is a straight chain alkyl group having from about 6 to about 20 carbon atoms, R² is a lower alkyl group having from about 1 to about 3 carbon atoms, preferably 1 carbon atom, and M is a water-soluble cation as described hereinbefore. Preferred anionic surfactants for use in the personal care compositions include sodium tridecyl benzene sulfonate, sodium dodecyl benzene sulfonate.

Amides, including alkanol amides, are the condensation products of fatty acids with primary and secondary amines or alkanolamines to yield products of the general Formula IX:

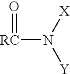

wherein RCO is a fatty acid radical and R is C₈₋₂₀; X is an alkyl, aromatic or alkanol (CHR′CH₂OH wherein R′ is H or C₁₋₆ alkyl); Y is H, alkyl, alkanol or X. Suitable amides include, but are not limited to cocamide, lauramide, oleamide and stearamide. Suitable alkanolamides include, but are not limited to, cocamide DEA, cocamide MEA, cocamide MIPA, isostearamide DEA, isostearamide MEA, isostearamide MIPA, lanolinamide DEA, lauramide DEA, lauramide MEA, lauramide MIPA, linoleamide DEA, linoleamide MEA, linoleamide MIPA, myristamide DEA, myristamide MEA, myristamide MIPA, Oleamide DEA, Oleamide MEA, Oleamide MIPA, palmamide DEA, palmamide MEA, palmamide MIPA, palmitamide DEA, palmitamide MEA, palm kernelamide DEA, palm kernelamide MEA, palm kernelamide MIPA, peanutamide MEA, peanutamide MIPA, soyamide DEA, stearamide DEA, stearamide MEA, stearamide MIPA, tallamide DEA, tallowamide DEA, tallowamide MEA, undecylenamide DEA, undecylenamide MEA. The condensation reaction may be carried out with free fatty acids or with all types of esters of the fatty acids, such as fats and oils, and particularly methyl esters. The reaction conditions and the raw material sources determine the blend of materials in the end product and the nature of any impurities.

Suitable optional surfactants include nonionic surfactants. Any such surfactant known in the art for use in hair or personal care products may be used, provided that the optional additional surfactant is also chemically and physically compatible with the essential components of the personal care composition, or does not otherwise unduly impair product performance, aesthetics or stability. The concentration of the optional additional surfactants in the personal care composition may vary with the cleansing or lather performance desired, the optional surfactant selected, the desired product concentration, the presence of other components in the composition, and other factors well known in the art.

Non limiting examples of other surfactants suitable for use in the personal care compositions are described in McCutcheon's, Emulsifiers and Detergents, 1989 Annual, published by M. C. Publishing Co., and U.S. Pat. Nos. 3,929,678, 2,658,072, 2,438,091, 2,528,378.

H. Optional Ingredients

1. Suspending Agent

The personal care compositions of the present invention may further comprise a suspending agent at concentrations effective for suspending the water-insoluble, dispersed material in the personal care compositions. Such concentrations range from about 0.1% to about 10%, preferably from about 0.3% to about 5.0%, by weight of the personal care compositions.

Suitable suspending agents include crystalline suspending agents that can be categorized as acyl derivatives, long chain amine oxides, or combinations thereof. These suspending agents are described in U.S. Pat. No. 4,741,855.

2. Anti-dandruff Actives

The compositions of the present invention may also contain an anti-dandruff agent. Suitable, non-limiting examples of anti-dandruff particulates include: pyridinethione salts, azoles, selenium sulfide, particulate sulfur, and mixtures thereof. Preferred are pyridinethione salts. Such anti-dandruff particulate should be physically and chemically compatible with the essential components of the composition, and should not otherwise unduly impair product stability, aesthetics or performance. These anti-dandruff actives are described in WO01/00151.

3. Humectant

The compositions of the present invention may contain a humectant. The humectants herein are selected from the group consisting of polyhydric alcohols, water soluble alkoxylated nonionic polymers, and mixtures thereof. The humectants, when used herein, are preferably used at levels by weight of the composition of from about 0.1% to about 20%, more preferably from about 0.5% to about 5%.

Polyhydric alcohols useful herein include glycerin, sorbitol, propylene glycol, butylene glycol, hexylene glycol, ethoxylated glucose, 1,2-hexane diol, hexanetriol, dipropylene glycol, erythritol, trehalose, diglycerin, xylitol, maltitol, maltose, glucose, fructose, sodium chondroitin sulfate, sodium hyaluronate, sodium adenosine phosphate, sodium lactate, pyrrolidone carbonate, glucosamine, cyclodextrin, and mixtures thereof.

Water soluble alkoxylated nonionic polymers useful herein include polyethylene glycols and polypropylene glycols having a molecular weight of up to about 1000 such as those with CTFA names PEG-200, PEG-400, PEG-600, PEG-1000, and mixtures thereof.

4. Miscellaneous

The compositions of the present invention may contain fragrance and/or herbal or botanical extracts.

The compositions of the present invention may also contain vitamins and amino acids such as: water soluble vitamins such as vitamin B1, B2, B6, B12, C, pantothenic acid, pantothenyl ethyl ether, panthenol, biotin, and their derivatives, water soluble amino acids such as asparagine, alanin, indole, glutamic acid and their salts, water insoluble vitamins such as vitamin A, D, E, and their derivatives, water insoluble amino acids such as tyrosine, tryptamine, and their salts.

The compositions of the present invention may also contain pigment materials such as inorganic, nitroso, monoazo, disazo, carotenoid, triphenyl methane, triaryl methane, xanthene, quinoline, oxazine, azine, anthraquinone, indigoid, thionindigoid, quinacridone, phthalocianine, botanical, natural colors, including: water soluble components such as those having C.I. Names.

The compositions of the present invention may also contain antimicrobial agents which are useful as cosmetic biocides and antidandruff agents including: water soluble components such as piroctone olamine, water insoluble components such as 3,4,4′-trichlorocarbanilide (trichlosan), triclocarban and zinc pyrithione. The compositions of the present invention may also contain chelating agents.

### Method of Making

The compositions of the present invention, in general, may be made by mixing the ingredients together at either room temperature or at elevated temperature, e.g., about 72° C. Heat only needs to be used if solid ingredients are incorporated into the composition. It is useful to fully hydrate the cationic conditioning polymer prior to addition of the microemulsified conditioning oil. The ingredients are mixed at the batch processing temperature. Additional ingredients, including electrolytes, polymers, and other optional ingredients, may be added to the product at room temperature.

### Method of Use

The personal care compositions of the present invention are used in a conventional manner for cleansing and conditioning hair or skin. An effective amount of the composition for cleansing and conditioning the hair or skin is applied to the hair or skin, that has preferably been wetted with water, and then rinsed off. Such effective amounts generally range from about 1 gm to about 50 gm, preferably from about 1 gm to about 20 gm. Application to the hair typically includes working the composition through the hair such that most or all of the hair is contacted with the composition.

This method for cleansing and conditioning the hair or skin comprises the steps of: a) wetting the hair or skin with water, b) applying an effective amount of the personal care composition to the hair or skin, and c) rinsing the applied areas of skin or hair with water. These steps can be repeated as many times as desired to achieve the desired cleansing and conditioning benefit.

### Non-Limiting Examples

The compositions illustrated in the following Examples illustrate specific embodiments of the compositions of the present invention, but are not intended to be limiting thereof. Other modifications can be undertaken by the skilled artisan without departing from the spirit and scope of this invention. These exemplified embodiments of the composition of the present invention provide enhanced deposition of the personal care composition due to enhanced coacervate formation.

The compositions illustrated in the following Examples are prepared by conventional formulation and mixing methods, an example of which is described above. All exemplified amounts are listed as weight percents and exclude minor materials such as diluents, preservatives, color solutions, imagery ingredients, botanicals, and so forth, unless otherwise specified.

The following are representative of clear shampoo compositions in accordance with the present invention.

| | Examples, wt % | | | | |
|---|---|---|---|---|---|
| Ingredient | 1 | 2 | 3 | 4 | 5 |
| Water | q.s. | q.s. | q.s. | q.s. | q.s. |
| Guar | 0.25 | — | — | — | — |
| Hydroxypropyltrimonium | | | | | |
| Chloride¹ | | | | | |
| Cationic Guar Polymer A² | — | 0.25 | — | — | — |
| Cationic Guar Polymer B³ | — | — | 0.25 | — | — |
| Cationic Guar Polymer C⁴ | — | — | — | 0.25 | — |
| Cationic Guar Polymer D⁵ | — | — | — | — | 0.25 |
| Sodium Laureth Sulfate | 29.31 | 34.48 | 34.48 | 24.14 | 17.24 |
| (SLE₃S - 29% active)⁶ | | | | | |
| Sodium Lauryl Sulfate | 18.97 | 27.59 | 34.48 | 24.14 | 31.03 |
| (SLS - 29% active)⁷ | | | | | |
| Dimethicone | 2.0 | 2.0 | 2.0 | 2.0 | 2.0 |
| Microemulsion⁸ | | | | | |
| PPG-2 Hydroxyethyl | 2.0 | 2.0 | 2.0 | 2.0 | 2.0 |
| Coco/Isostearamide⁹ | | | | | |
| Sodium Chloride¹⁰ | 1.2 | 1.2 | 1.2 | 1.2 | 1.2 |
| Fragrance | 0.75 | 0.75 | 0.75 | 0.75 | 0.75 |
| Preservatives, pH adjusters | Up to 1% | Up to 1% | Up to 1% | Up to 1% | Up to 1% |
| Calculated: | | | | | |
| Ethoxylate level | 2.50 | 2.94 | 2.94 | 2.06 | 1.77 |
| Sulfate level | 3.07 | 4.02 | 4.55 | 3.19 | 3.00 |

¹N-Hance 3205, MW = 1 MM, charge density = 0.8 meq/gram, supplier Hercules (Aqualon)
²Experimental cationic guar polymer with MW = 1 MM and charge density = 1.6
³Experimental cationic guar polymer with MW = 1 MM and charge density = 2.1
⁴Experimental cationic guar polymer with MW = 600 M and charge density = 1.6
⁵Experimental cationic guar polymer with MW = 450 M and charge density = 1.6
⁶Sodium Laureth Sulfate at 29% active with an average of approximately 3 moles of ethoxylation, supplier: P&G
⁷Sodium Lauryl Sulfate at 29% active, supplier: P&G
⁸DC2-1870, 30 nm particle size dimethicone using TEA dodecyl benzene sulfonate and POE lauryl ether as primary surfactants, supplier Dow Corning
⁹Promidium 2, supplier Uniqema
¹⁰Sodium Chloride USP (food grade), supplier Morton.

The following are representative of clear shampoo compositions containing secondary conditioning ingredients as well as additional surfactants:

| | Examples, wt % | | | | | | | |
|---|---|---|---|---|---|---|---|---|
| Ingredient | 6 | 7 | 8 | 9 | 10 | 11 | 12 | 13 |
| Water | q.s. | q.s. | q.s. | q.s. | q.s. | q.s. | q.s. | q.s. |
| Guar | 0.20 | 0.20 | 0.20 | — | — | — | — | 0.20 |
| Hydroxypropyltrimonium | | | | | | | | |
| Chloride¹ | | | | | | | | |
| Cationic Guar Polymer A² | — | — | — | 0.20 | 0.20 | 0.20 | 0.10 | — |
| Sodium Laureth Sulfate | 29.31 | 29.31 | 29.31 | 34.48 | 34.48 | 34.48 | 34.48 | 29.31 |
| (SLE₃S - 29% active)³ | | | | | | | | |
| Sodium Lauryl Sulfate | 18.97 | 18.97 | 18.97 | 27.59 | 27.59 | 27.59 | 27.59 | 18.97 |
| (SLS - 29% active)⁴ | | | | | | | | |
| Dimethicone | 2.0 | 4.0 | 8.0 | 8.0 | 8.0 | 8.0 | 8.0 | 8.0 |
| Microemulsion⁵ | | | | | | | | |
| TEA Dodedeyl Benzene | — | — | — | — | — | — | — | 2.0 |
| Sulfonate⁶ | | | | | | | | |
| POE Lauryl Ether⁷ | — | — | — | — | — | — | — | 0.35 |
| Cocamidopropyl Betaine⁸ | 7.0 | 7.0 | 7.0 | 7.0 | 7.0 | 3.33 | 3.33 | 7.0 |
| Disodium | — | — | — | — | — | 2.0 | 2.0 | — |
| Cocoamphodiacetate⁹ | | | | | | | | |
| PPG-2 Hydroxyethyl | 2.0 | 2.0 | 2.0 | 2.0 | 2.0 | 2.0 | 2.0 | 2.0 |
| Coco/Isostearamide¹⁰ | | | | | | | | |
| Cetyl Alcohol¹¹ | 0.50 | — | — | — | — | — | — | — |
| Di-PPG-2 Myreth-10 | — | — | — | — | 2.0 | — | 1.0 | 2.0 |
| Adipate¹² | | | | | | | | |
| Linoleamidopropyl PG- | — | — | — | 2.0 | 2.0 | — | — | 2.0 |
| Dimonium Chloride | | | | | | | | |
| Phosphate¹³ | | | | | | | | |
| Magnesium Chloride, | 0.50 | 0.50 | 0.50 | 0.50 | 0.50 | 0.50 | 0.50 | 0.50 |
| hexahydrate¹⁴ | | | | | | | | |
| Sodium Chloride¹⁵ | 1.0 | 1.2 | 1.2 | 0.7 | 0.8 | 0.9 | 1.1 | 1.0 |
| Fragrance | 0.75 | 0.85 | 0.85 | 0.85 | 0.85 | 0.85 | 0.85 | 0.85 |
| Preservatives, pH adjusters | Up to 1% | Up to 1% | Up to 1% | Up to 1% | Up to 1% | Up to 1% | Up to 1% | Up to 1% |
| Calculated: | | | | | | | | |
| Ethoxylate level | 2.50 | 2.50 | 2.50 | 2.94 | 2.94 | 2.94 | 2.94 | 2.50 |
| Sulfate level | 3.07 | 3.07 | 3.07 | 4.02 | 4.02 | 4.02 | 4.02 | 3.07 |

¹N-Hance 3205, MW = 1 MM, charge density = 0.8 meq/gram, supplier Hercules (Aqualon)
²Experimental cationic guar polymer with MW = 1 MM and charge density = 1.6
²Sodium Laureth Sulfate at 29% active with an average of approximately 2 moles of ethoxylation, supplier: P&G
³Sodium Laureth Sulfate at 29% active with an average of approximately 3 moles of ethoxylation, supplier: P&G
⁴Sodium Lauryl Sulfate at 29% active, supplier: P&G
⁵DC2-1870, 30 nm particle size dimethicone using TEA dodecyl benzene sulfonate and POE lauryl ether as primary surfactants, supplier Dow Corning
⁶Biosoft N-300 (60% active), supplier Stepan
⁷BRIJ 35, Supplier Uniqema
⁸Tegobetaine F-B (30% active), supplier Degussa
⁹Miranol C2M Conc NP (50% active), supplier Stepan
¹⁰Promidium 2, supplier Uniqema
¹¹CO-1695F, supplier P&G
¹²Cromollient SCE, supplier Croda
¹³Phospholipid EFA (30% active), supplier Uniqema
¹⁴Magnesium Chloride 6-Hexahydrate, supplier Fisher Chemicals
¹⁵Sodium Chloride USP (food grade), supplier Morton.

The following are representative of body wash compositions of the present invention.

| | Example, wt % | | | |
|---|---|---|---|---|
| Ingredient | 14 | 15 | 16 | 17 |
| Water | q.s. | q.s. | q.s. | q.s. |
| Guar Hydroxpropyltrimonium Chloride¹ | 0.1 | | | 0.1 |
| Guar Hydroxpropyltrimonium Chloride² | | 0.1 | 0.1 | |
| Sodium Laureth Sulfate (SLE3S)³ | 29.31 | 34.48 | 34.48 | 29.31 |
| Sodium Lauryl Sulfate⁴ | 18.97 | 27.59 | 27.59 | 18.97 |
| Silicone Microemulsion⁵ | 2.0 | | 1.0 | 2.0 |
| Cetyl Alcohol⁶ | | 0.5 | | |
| Disodium Laureth Sulfosuccinate⁷ | 1.0 | | | |
| Glycerine⁸ | 1.0 | | 2.0 | |
| Cocamidopropyl Betaine⁹ | 3.0 | | | |
| Disodium Coco Amphodiacetate¹⁰ | | 4.00 | 1.0 | |
| Decyl Glucoside¹¹ | | 1.0 | | |
| Glyceryl Stearate, Cetearyl Alcohol, | | | 0.5 | |
| Stearic Acid, 1-Propanamimium-3-Amino- | | | | |
| N-(2-hydroxyethyl)N,N-Dimethyl-N-C16- | | | | |
| 18 Acryl Derivs., Chlorides¹² | | | | |
| Perfluoropolymethylisopropyl Ether¹³ | | | | 1.0 |
| Magnesium Chloride, hexahydrate¹⁴ | 0.5 | | | |
| Sodium Chloride¹⁵ | 1.4 | 1.2 | 1.2 | 2.0 |
| Fragrance | 0.55 | 0.55 | 0.55 | 0.55 |
| Preservatives, pH adjusters | <1.0 | <1.0 | <1.0 | <1.0 |
| Calculated levels based on surfactants | | | | |
| added above: | | | | |
| Ethoxylate level | 2.50 | 2.94 | 2.94 | 2.50 |
| Sulfate level | 3.07 | 4.02 | 4.02 | 3.07 |

¹N-Hance 3205, MW = 1 MM, charge density = 0.8 meq/gram, supplier Hercules (Aqualon)
²Experimental cationic guar polymer with MW = 1 MM, charge density = 1.6 meq/gram
³Sodium Laureth Sulfate at 29% active with an average of approximately 3 moles of ethoxylation, supplier: P&G
⁴Sodium Lauryl Sulfate at 29% active, supplier: P&G
⁵DC2-1870, 30 nm particle size dimethicone using TEA dodecylbenzene sulfonate and POE lauryl ether as primary surfactants, supplier Dow Corning.
⁶CO-1695, supplier P&G
⁷Stepan-MILD LSB, supplier Stepan
⁸Star, supplier: Procter & Gamble
⁹Tegobetaine (30% active), supplier Goldschmidt (Degussa)
¹⁰Miranol C2M Conc NP (50% active), supplier Rhodia
¹¹Plantaren PS-100, supplier Cognis Care Chemicals
¹²Prolipid 151, supplier ISP
¹³Fomblin HC/04, supplier Ausimont
¹⁴Magnesium Chloride 6-Hexahydrate, supplier Fisher Chemicals
¹⁵Sodium Chloride USP (food grade), supplier Morton.

All documents cited in the Detailed Description of the Invention are, are, in relevant part, incorporated herein by reference; the citation of any document is not to be construed as an admission that it is prior art with respect to the present invention.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A clear personal care composition comprising:
a. from about 0.01 to about 5 wt. % of a cationic guar polymer, wherein said cationic guar polymer has a molecular weight of at least 300,000 and a charge density of at least about 0.1 meq/g;
b. from about 5 to about 50 wt. % of an anionic surfactant system having an ethoxylate level and a sulfate level, wherein said ethoxylate level is in the amount of about 1.97 multiplied by the molecular weight of said cationic guar polymer divided by 1,000,000 plus from about 0.15 to about 3.5, and further wherein said sulfate level is in the amount of about 0.97 multiplied by the charge density of said cationic guar polymer plus from about 1 to about 4,
c. from about 0.01 to about 5 wt. % of a mono or divalent salt;
d. from about 0.01 to about 10 wt. % of a microemulsified conditioning oil having a droplet size of less than about 80 nm;
e. from about 0.05 to about 5% of a secondary conditioner; and
f. at least about 20 wt. % of an aqueous carrier;
wherein the composition, prior to the addition of any colorants and/or pigments, has a percent transmittance at 600 nm of at least about 65%.

2. A personal care composition according to claim 1 wherein said microemulsified conditioning oil is selected from the group consisting of silicone oils, hydrocarbons oils, polyolefins, fatty esters, fluorinated compounds and mixtures thereof.

3. A personal care composition according to claim 1 wherein said cationic guar polymer has a molecular weight of at least about 1,000,000.

4. A personal care composition according to claim 1 wherein said cationic guar polymer has a charge density of at least about 0.5 meq/gm.

5. A personal care composition according to claim 1 wherein said cationic guar polymer is present in a concentration of from about 0.05 wt. % to about 2.0 wt. %.

6. A personal care composition according to claim 1 wherein said cationic guar polymer is in a coacervate phase or forms a coacervate phase upon dilution.

7. A personal care composition according to claim 1 wherein said anionic surfactant system is selected from the group consisting of alkyl sulfates, alkyl ether sulfates, and mixtures thereof.

8. A personal care composition according to claim 1 further comprising zwitterionic or amphoteric surfactants in a concentration of from about 0.5 wt. % to about 20 wt. %.

9. A personal care composition according to claim 1 further comprising zwitterionic or amphoteric surfactants in a concentration of from about 1 wt. % to about 5 wt. %.

10. A personal care composition according to claim 1 wherein said mono or divalent salt is selected from the group consisting of chlorides, phosphates, sulfates, nitrates, citrates, halides, and mixtures thereof.

11. A personal care composition according to claim 1 wherein said mono or divalent salt is present in a concentration of from about 0.05 wt. % to about 3.5 wt. %.

12. A personal care composition according to claim 1 wherein said secondary conditioning agent is selected from the group consisting of quaternary ammonium compounds, fatty esters, fatty alcohols, polyethylene glycols, alkyl glucosides, alkyl glucoside derivatives and mixtures thereof.

13. A method of treating hair or skin said method comprising the steps of
a) wetting the hair or skin with water;
b) applying an effective amount of a personal care composition according to claim 1, to the hair or skin; and
c) rinsing said hair or skin with water.
